(19) United States
(12) Patent Application Publication
(10) Pub. No.: US 2004/0236383 A1
(43) Pub. Date: Nov. 25, 2004

(54) MONES for muscle strain, repetitive strain injuries and related health conditions

(76) Inventor: Nikolay I. Yelizarov, Richmond, BC (CA)

Correspondence Address: NIKOLAY YELIZAROV, #82--10200 4TH AVE, RICHMOND, BC, V7E 1V3, CA

(21) Appl. No.: 10/249,975

(22) Filed: May 23, 2003

(51) Int. Cl. A61N 1/18

(52) U.S. Cl. 607/48

(57) ABSTRACT

A set of electrically induced muscle contractions serves the purpose of rapid recovery of muscle strain injury and related health problems such as low back pain and sciatica, upper neck pain and headache or dizziness etc. A single muscle contraction is induced by a single impulse between tips of two electrodes (acupuncture needles) by employing lower motor neuron path way. Such purpose performs by stationary placed of one needle on a vertebra (periosteum) and another one inside injured muscle. The impulse goes through dorsal root—lower motor neuron—muscle and induces whole muscle contraction. A set of such contractions with frequency between 1-2 Hz pumps blood and lymph through the tissue, makes greater the pressure gap between arterial and venous blood. Therefore reduced swelling, restored blood circulation and released from compression intramuscular nerve improve patient feelings on long term. Due to complexity of muscle strain injury author offers two additional treatments neurotome and osteotome electrical stimulation.

### CROSS REFERENCE TO RELATED APPLICATIONS

Current U.S. Class: 607/48 Intern'l Class: A61N 001/36 Field of Search: 128/898, 600/598, 604/154, 606/185, 189, 607/2, 3, 46, 48, 50, 62, 63, 69, 115 U.S. patent Documents: U.S. Pat. No. 4,180,079 December, 1979 Wing 607/69 U.S. Pat. No. 4,276,879 July, 1981 Yiournas 604/154 U.S. Pat. No. 4,699,143 October, 1987 Dufresne et al. 607/46 U.S. Pat. No. 4,759,368 July, 1988 Spanton et al. 607/46 U.S. Pat. No. 4,832,032 May, 1989 Schneider 607/115 U.S. Pat. No. 4,989,605 February, 1991 Rossen. 607/46 U.S. Pat. No. 5,041,974 August, 1991 Walker et al. 607/63 U.S. Pat. No. 5,183,041 February, 1993 Toriu et al. 607/46 U.S. Pat. No. 5,211,175 May, 1993 Gleason et al. 600/548 U.S. Pat. No. 5,304,207 April, 1994 Stromer 607/3 U.S. Pat. No. 5,350,414 September, 1994 Kolen 607/62 U.S. Pat. No. 5,354,320 October, 1994 Schaldach et al. 607/46 U.S. Pat. No. 5,374,283 December, 1994 Flick. 607/46 U.S. Pat. No. 5,387,231 February, 1995 Sporer 607/48 U.S. Pat. No. 5,395,398 March, 1995 Rogozinski 607/50 U.S. Pat. No. 5,397,338 March, 1995 Grey et al. 607/115 U.S. Pat. No. 5,476,481 December, 1995 Schondorf. 607/2 U.S. Pat. No. 5,735,868 April, 1998 Lee 606/189 U.S. Pat. No. 5,968,063 Oct., 19, 1999 Chu, et al. 606/185 U.S. Pat. No. 6,058,938 May, 2000 Chu, et al. 128/898

### BACKGROUND OF INVENTION

Muscle injuries described in this work are muscle-strain injuries (MSI) and repetitive strain injuries (RSI). MSI and RSI are frequently associated with work-related muscular-skeletal disorders (WMSD). Strain injuries include those to the bone, ligament, tendon, joint-cartilage, synovial tissue, muscle, vascular system, and nervous tissue. Strain injuries are often related to a certain type of work activity, poor posture, biomechanics, or lifestyle activity.

Strain injury (SI) is a common name for conditions such as cumulative trauma disorders, repetitive strain injuries, typing injuries, and related conditions like carpal tunnel syndrome, trigger-finger, tennis-elbow, whiplash, etc.

Strain injuries can be divided into two classes:

1) Acute injury due to excessive force being applied to the muscle when lifting or falling.

2) Chronic injuries due to excessive repetition, as in typing.

MSI describes injuries to the soft tissues, which occur due to excessive force being applied to the muscle(s). RSI, or cumulative trauma disorder, describes injuries to the soft tissues that are caused, over time, by repetitive actions.

Muscle strain injuries (MSI) are related to acute muscle strain injuries, such as whiplash, and repetitive strain injuries (RSI) are related to chronic-type injuries. RSI is a result of the muscles being in a high-alert mode during repetitive movements, which stress these in-use muscles. Small repetitive movements can injure muscles as well as tendons. Programmers, computer users, hairstylists, dentists, cooks, etc. often acquire wrist or shoulder problems due to prolonged repetitive motion of the same muscles. A person may suffer for several months, or even years, often bringing down their quality of life.

MSI are associated with isotonic contraction, where there is actual physical damage to muscle cells. With RSI, there is isometric contraction and impairment of the blood flow. There is an imbalance between the blood demand and supply. An enhanced blood flow would create a positive impact in both conditions.

Strain Injuries pain-sensitivity based classification:

â□Muscle injury, up to 10% of muscle diameter, is unnoticed, no pain.

â□Muscle injury, up 10-30% of muscle diameter, is painful during palpation.

â□Muscle injury, up more than 30% across the muscle distance, will make the patient complain about muscle pain and movement limitations.

The depth of the injury is more important than the length because of the impact on muscle performance. In addition, that is why the distal end of muscle is more vulnerable. The across-distance of the vastus medialis, for example, is much greater than at the insertion of the muscle (patella).

There is a tendency to frame a patient's complaints into syndromes, which allow for collecting statistics by tracking the incidence among occupations. Unfortunately, we know more about statistics than we do about the nature of strain injuries.

“In 1996/97, nearly two million Canadians, aged 12 and older, sustained repetitive strain injuries (RSI) that were serious enough to reduce their usual activities. These injuries, caused by overuse of certain muscles, included carpal tunnel syndrome, tennis-elbow, tendonitis, and back injury. Injuries to the back or spine accounted for the greatest share (20%) of RSI among men. Injuries of the wrist, hand, or fingers were the most common among women (25%).”(National Population Health Survey: Cycle 2, 1996/97).

The management of chronic pain due to repetitive strain injuries was a $95 billion dollar business in the United States in 2000 according to the U.S. Occupational Safety and Health Administration (OSHA) estimations. These numbers indicate that strain injuries are widespread and costly, and can last for years, and surgery is often necessary.

There are a few non-surgical approaches to the MSI/RSI/WMSD problems, but every approach has limitations that reduce its efficiency.

Drugs: Painkillers, anti-inflammatory medication, and muscle relaxants

Â Painkillers help to control the pain level and slow the inflammation; the recurrence of pain is high after returning the patient to the same work or lifestyle. In addition, because of instant pain relief, the proper treatment will be postponed, sometimes leading to overuse of these drugs. Painkillers should only be used for emergency help. Anti-inflammatory medication: hormone injections, sometimes called, “despair therapy,” suppresses inflammation activity and body immune responses, and reduces capillary permeability. The mechanism of hormone actions is not yet fully understood.

Â Muscle relaxants block inter-neuronal activity in the descending reticular formation and spinal cord. Muscle relaxants increase muscle-pain-threshold and reduce muscle response to rehabilitation therapy (electrical stimulation, exercises). Some muscle relaxants cause withdrawal effects.

Another side effect of drugs is a pain reduction as well. It sounds like a paradox but, if the patient will take a medication and do not take a rest. He/she will cause more the harm than a without a medication.

Massage improves blood and lymph circulation, and increases metabolism in the superficial muscles. However, the masseur's fingers cannot access deep muscles. Massage of overweight patients is problematic even for superficial muscles. In addition, massage of the injured muscle can be very painful, and is not recommended during the early stage of injury (the first 10-14 days), because of the possibility of worsening the injury.

TENS electrical stimulation is commonly used for MSI treatment.

The principle is patch's placements in boundaries of a myotome. Two and more conductive patches apply in one session at once with high frequency for pain relief purpose. The effectiveness of the treatment is short term and reduced significantly by the layer of fat under the skin. Both skin and adipose tissues are rather insulators than conductors; therefore, a weakened electrical impulse can only reach superficial muscles. Latest research (Journal of Physical Therapy) tells us that there is no significant difference between TENS and exercise. We believe that because the main goal of TENS is analgesia.

Acupuncture-like TENS (AL-TENS) is an electro-acupuncture combining TENS″ principles, like needle insertion in boundaries of one myotome, and low frequency current. The purpose is the same as TENS, to produce analgesia.

In the last few years, more research has been done on efficacy of TENS and AL-TENS. The conclusion is that over a period of three to six months TENS and AL-TENS are not effective for low back pain.

Exercise programs are not recommended during an acute stage of MSI, but are definitely needed during the rehabilitation stage. Exercise improves the overall blood and lymph circulation. Stretching is the most popular exercise for MSI, but improper or very extensive exercise can create additional injury and pain. Exercise only, can not cure MSI.

Chiropractors use spine alignment to treat low back pain and sore neck The approach is right; in some cases pain is created by one or a few nerves pinched by the out-of-alignment vertebrae. Proper spine alignment is provided when muscles are symmetrically attached to the vertebra. If a muscle from one side is swollen and shortened, it will pull the vertebra to the side. It is clear that the proper spine alignment cannot be achieved without treating the shortened muscle. Chiropractic patients benefit more from combined muscle treatment as well as spine alignment.

Acupuncture has a few very important advantages:

a) Direct treatment

b) May reach a muscle, no matter how deep

c) Uses no medication

d) No side effects

Disadvantage of acupuncture: Since all acupuncture schools teach diagnostic tools, not every practitioner is able to practice without a “recipe book.” It decreases the value of acupuncture and sometimes discredits it.

Besides the above-mentioned MSI treatments, other methods that use acupuncture principals and needles should be noticed.

Â Dr. Gunn's intra-muscular stimulation (IMS) is a treatment for muscle spasm, or shortened muscle, by desensitizing the muscle trigger-point. The treatment is based on a neurological approach to the problem. Dr. Gunn states that the injured nerve is hypersensitive (Cannon and Rosenblueth' Law of Denervation) and can be desensitized by needle insertion and manipulation. A needle is inserted into the spasm area and followed by needle grasp and muscle's fasciculation. Visually, it appears as a muscle twitch. The treatment requires a needle manipulation on each shortened muscle. It is difficult to employ the technique on shortened deep muscles near the vertebra, under blind control. The treatment is quite painful.

Â Dr. Chu (U.S. Pat. No. 6,058,938, et al. May 9, 2000) found that the acu-needle inserted only once into the muscle does not provide the necessary stimulation. She offers twitch-obtaining intramuscular electrical stimulation, which is performed by motor end-plate employment. The rate of stimulation is 10-50 Hz. Both electrodes (Teflon-coated needles) are placed, subcutaneous, about 15-25 mm away from each other. It elicits “twitches in a small portion of muscle, visible as fine jerking of the stimulating needle or as fasciculation-like twitches.” This is very effective and cost-efficient technique, but produces only local intra-muscle stimulation. To treat the whole muscle, the needle insertion has to be repeated in at least four treatment points within the afflicted muscle. Note: the repetitive needling can create stress for some patients.

### SUMMARY OF INVENTION

In view of the foregoing, the principal object of the present invention is to provide a simplified and standardized treatment that medical personnel, including but not limited to doctors, can rapidly be trained on, thus making the treatment available at low cost and on a mass scale. This will lead to reduced absenteeism from work, payoff for worker's compensation, and disability expenses. In addition, a larger number of pain afflicted persons may enjoy happier and more productive and fulfilling lives.

The further object of the invention is to improve the effectiveness of recent myotherapy techniques, i.e., to increase and prolong the resultant pain relief, and reduce tissue trauma and patient discomfort associated with the IMS procedure.

These and other objects are achieved in accordance with the present invention by a method of conducting an electrical intramuscular stimulation through lower motor neuron therapy session.

There are three variations of the modality:

1) osteotome treatment

2) myotome treatment

3) neurotome treatment

The commonalties between these three variations are:

a) Electrode's location that used for stimulating of a treatment area: one needle always placed on a vertebra (periosteum) and second needle or electro-conductive patch in distal area of nerve distribution.

b) Electrodes' polarity: (−) for vertebra and (+) for second electrode.

c) The electrical impulse frequency: 1 to 2-2.5 Hz serves the purpose of microcirculation rehabilitation.

d} The voltage lays in more broad numbers than frequency - - - 5 mAmp (some neck muscles) to 55 mAmps. The voltage number depends on precise needling the periosteum, the distance between two electrodes and nerve diameter between two electrodes.

1) Osteotome 3electrical Stimulation

Treatment is applied to a vertebra which is a sensitive to finger pressure. Intrinsic muscles, tendons, and other soft tissues that are in direct contact with the vertebra compose an osteotome. Osteotome structures can be a source of pain and have to be treated. The (−) electrode (needle) is placed on the vertebra and second (+) electrode (patch in this case),on a stomach. It is very important that the tip contacts periosteum. The greater electrical charge concentrates on a tip of a needle and therefore provides stimulation to tissues surrounding vertebra. The patch supports the current direction from (−) to (+) and it must be placed within the boundaries of area of nerve distribution of the vertebra. The treatment provides easy and effective stimulation for unattainable structures around vertebra: tendons, nerves, muscles, bones, and vessels.

FIG. 1: Osteotome Electrical Stimulation.

2) Myotome Electrical Stimulation:

Myotome treatment aims to release a pressure on the inside injured muscle nerve created by inter-muscular oedema (intramuscular nerve compression)At first, recognition of sore muscle is based on the movement restriction or pain provoking movement. Secondly, the vertebra responsible for nerve distribution in the injured muscle has to be recognized as well. An acupuncture needle is inserted into the muscle's tissue in such manner that the tip of the needle is placed approximately in the middle of the muscle. The second acupuncture needle places on a vertebra contacting periosteum. The vertebra and the muscle should be in the boundaries of the same myotome. The main idea is based on a concept that electrical current always goes by the best conductivity way. Therefore the closer needle's tips is placed to a nerve the lesser voltage is required.

FIG. 2: Myotome electrical stimulation

Explanation. The distance between needles must be grate to obtain the more chances for impulse to go through the nerve. The mayor challenge is vertebra needle; it must be placed on right spot (periosteum) which is almost in intimate contact with a spinal nerve. Therefore the current will go through nerve pathway directly to the tip of second needle which is placed into muscle nerve net and stimulate the whole muscle. The current flows from the tip of vertebra needle to periosteum to spinal nerve to a muscle and to a second needle. The muscle needle is withdrawn from the muscle upon 7-10 min of the stimulation. The foregoing steps are repeated to elicit contraction at as many muscles as required upon a limit of time, boundaries of myotome and common sense.

3) Neurotome Electrical Stimulation

A pain patient suffer may come from a major nerve compressed between muscles (sciatic) or because of narrowing the space where it goes trough (median nerve trough carpal tunnel). Compressed nerve causes a lot of pain and discomfort at both scenarios. Removal of the compression is a main goal of the treatment. The electrodes have to be place on a beginning of the nerve and the distal area of the nerve to make sure the impulse will go trough the nerve. One needle (−) electrode is placed on a periosteum of vertebra responsible for the nerve distribution and (+) electrode, (electro-conductive patch) attaches on a skin on a distal part of the nerve, like calf, foot or hand, preferably on a spot of short distance between nerve branch and skin. The electrode placement will provide stimulation to nerve and other structures contacting the nerve. An impulse with the frequency will reduce the muscle and nerve swelling; restore microcirculation and create the necessary room for nerve pathway. The essential element of the procedure is to recognize involved vertebra and the level of compression. The patch must be placed far below the compression level.

FIG. 3: Neurotome Electrical Stimulation

The above and other objects, features, and advantages of the present invention will be readily apparent and fully understood from the following detailed description of preferred embodiments, taken in connection with the appended drawings

### BRIEF DESCRIPTION OF DRAWINGS

FIG. 1: Osteotome Electrical Stimulation. The needle is placed in touch with impaired vertebra's periosteum. The patch is placed on a median line of the abdominal wall within boundaries of the spinal nerve distribution, basically on a vertebra's cross sectional line and frontal median line. Wires are attached: (−) to a vertebra's needle and (+) to a patch on a stomach wall.

FIG. 2: Myotome Electrical Stimulation. The figure is a highly simplified schematic view of an electrical field distribution when needle #1 is placed on a vertebra's periosteum and needle #2 in boundaries of nerve distribution of the vertebra. The current flows through the periosteum to the spinal nerve to the nerve and to the muscle. Negative polarity is provided to #1 needle and positive polarity to #2 needle by any TENS or electro acupuncture device as soon the device can provide a single monophasic/biphasic impulse with a frequency 1-2,5 Hz and intensity in a range of 5-50 mAmps. Patients must feel no irritations but powerful contractions because of efficient conductive way of electrical impulse distribution between two needles. The first needle is placed in touch with impaired vertebra's periosteum. The second needle is placed into the muscle, the source of pain

FIG. 3: Neurotome Electrical Stimulation. The needle is placed in touch with impaired vertebra's periosteum. The patch is placed in a distal area of involved nerve branch. It supposed to be the area where the distal part of the nerve comes close to skin. Example: In a sciatica case, it is the area of distal lateral side of gastrocnemius muscle. In a carpal tunnel syndrome case, it is a base of the wrist on a palmar side

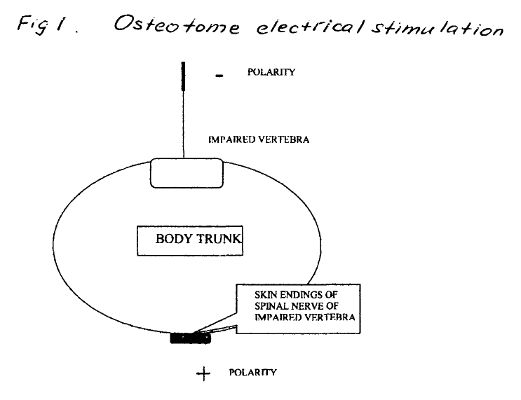

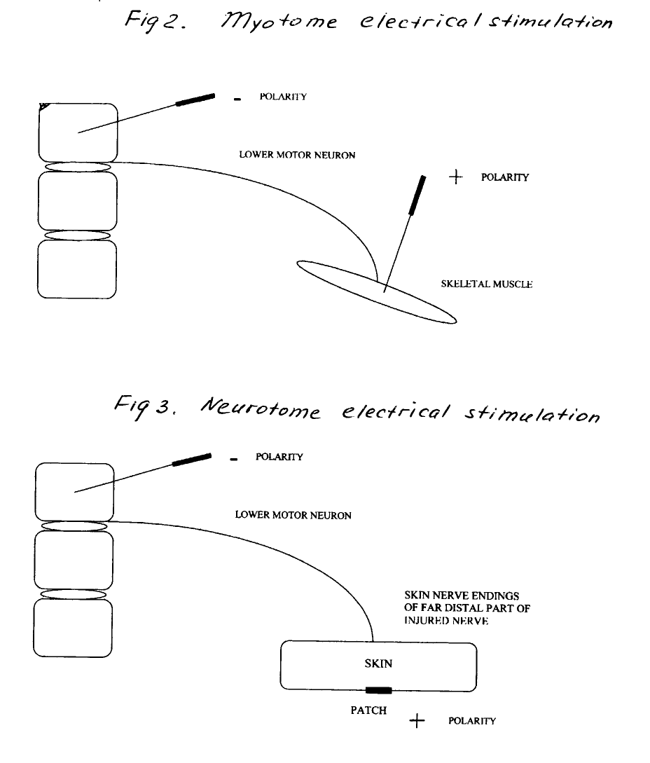

### DETAILED DESCRIPTION

General Principles

1.A sound knowledge of anatomy is essential. The trainee is referred to the textbook Gray's Anatomy of the Human Body (any edition) and to the physician/inventor's textbook Nikolay Yelizarov: Treatment for Muscle Strain Injuries. ISBN 1-932303-07-03.

The treatment program got a chose abbreviation as MONES (myotome, osteotome, neurotome electrical stimulation).

Myotome Electrical Stimulation (MES).The main goal of any treatment is to bring the healing factor, as directly as possible, to the injured organ. The most beneficial treatment comes from the precise delivery of the treatment. This rule is applied to any field of medicine. Therefore, it applies to muscle-strain injury. The best tools to provide this healing are the acupuncture needle and inducing muscle contraction in a solitary metamere. (A needle can reach almost any skeletal muscle). The treatment, within metamere boundaries, will rehabilitate all noticed and unnoticed injuries.

Muscle contraction will restore impaired microcirculation. The power of induced muscle contraction will give feedback for correct placement of the needle. Successful contraction of idle muscles will help the edema and restore microcirculation-the necessary recovery tool that was discussed above.

The muscle venous-blood circulation is performed by muscle contraction. Often an injured muscle is unable to obtain sufficient contraction to move the blood out due to some ruptured muscle threads; edema is most often the cause of the pain—pinched nerve fibers between swollen muscle threads or muscles. So every contraction (like simple exercise), creates more excessive pressure inside the muscle, and therefore, pain, which limits the movement. A single electrical impulse sent to a single injured muscle through a natural pathway (lower motor neurons) will create a like physiological contraction which will be sufficient enough for good blood circulation inside the muscle.

The most difficult point for treatment is to employ the lower motor neuron. Two needles should be used to obtain muscle contraction. An electrical impulse follows physics' law and takes the path of least resistance. Two needles inserted inside the same single muscle, or within boundaries of the myotome, will stimulate a part of the muscle by employing only a single part. Usually, especially in long muscles, the injured part is presented through the length of almost the entire muscle. Therefore, it is a complicated process to insert two needles at the same depth in the same muscle, especially if the muscle is covered by another muscle. In addition, there are always a few injured spots inside one muscle, making the procedure more complicated and painful. Also, patients will always tell the practitioner about uncomfortable feeling around the needle(s) because of the electrical irritation. The stimulation has to be general and direct.

To effectively stimulate the muscle, we have to use a lower motor neuron. This is the best way to produce conductivity within the body's system. Again, the difficult point is the implementation itself. For many reasons, we cannot insert a needle into the nerve pathway, particularly because of the risk of pain and nerve damage. We have to employ the nerve without touching it. Therefore, we need a mediator for it, and a vertebra suits serves this purpose. It is a large structure and is in intimate contact with the nervous system. It is very superficial, is a good conductor of electricity because of the countless contacts between the periosteum and spinal nerves, and it does not have pain receptors.

Another subtlety is when a needle placed on a vertebra and a second needle, or patch, supposes to employ a specific nerve pathway. It means they have to work in the same metamere. When needles, one on a vertebra and another on the muscle, are established, and stimulation is applied, the impulse goes through the periosteum, nerve root, and nerve branches, then directly to the injured muscle, and stimulates it. If the pathway is well established, the stimulation is effective and painless. The intensity does not have to be great, just efficient enough for the obvious muscle-contraction. The numbers are below: Frequency of the stimulation is important. We know from experience of other practitioners, and many articles about electro-acupuncture, that low frequency stimulates, and high frequency suppresses. For example, Chinese anesthesiologists employ high-frequency electrical stimulation for local anesthesia (suppression of nerve conductivity), with great success. TENS at physiotherapy, uses high frequency, 20-49 Hz for analgesia, AL-TENS uses a set of impulses with low frequency, two-four Hz (burst mode). We believe that only low frequency will support the idea of restoring of blood microcirculation. The relaxation time must be much greater than a time of contraction. Contraction induced by single electrical impulse pushes the blood out the muscle like a wringing and creates pressure gap necessary for bringing arterial blood in the muscle. Most suitable frequency is one-three Hz. Also, it supposes to be a normal mode, single impulse.

Neurotome Electrical Stimulation (NES).

Nerve's anatomy. As mentioned above, we call the metamere an anatomical structure, consisting of dermatom (skin), myotome (muscle), neurotome (nerve) and osteotome (bone). Since dermatom and myotome are more familiar, neurotome, and especially osteotome are not. When we talk about osteotome, we mean mostly the vertebra's periosteum, which is richer on nerve endings, and has more links to the nerve network than does bone.

Neurotome is more complicated than other parts of the metamere. It consists of the central nervous system (spinal cord) and autonomic nervous system. One of the principal functions of the spinal cord is to serve as the center for reflex actions. Spinal nerves are paths of communication between the spinal cord and the rest of the body. Each of the 31 pair of spinal nerves is connected to the spinal cord by two roots. The anterior, or ventral root, conducts impulses, via motor neurons, to muscular-skeletal systems, and anterior or visceral, brings information to the spinal cord via sensory fibers. The dorsal root of each spinal nerve has a spinal ganglion, which is located in the intervertebral foramen. Distal to the spinal ganglion, and just outside the intervertebral foramen, the dorsal and ventral nerve roots unite to form a spinal nerve, which divides almost immediately into ventral ramus (viscera) and dorsal ramus (muscles). The autonomic nerve system is made be both a sympathetic and parasympathetic nervous system. The characteristic of pain is different for the central nervous system and autonomic nervous system.

Table

| Peculiarity of clinical | Visceral pain | |
|---|---|---|
| picture | (ANS) | Somatic pain |
| Pain sensation | Smarting pain | Nagging, aching, |
| | | shooting, pulsing |
| Feeling”s location | Diffusely spreads | Definite location, |
| | | certain area |
| Permanency of location | Migrates | Does not |
| | | migrates |
| Area of primary | Cannot always tell | Always clearly |
| appearance | | determined |
| Area of referred pain | Determination is | Determination is |
| | difficult | easy |
| Referred pain to reflex | Yes | No |
| zone | | |
| Repercussion | Yes | No |
| Reflex impairment | Visceral reflex | Somatic reflex |
| | impairment | impairment |
| Light irritation | Negative | May distract the |
| | influence pain | pain |
| | increase | |
| Sore spots | Around vessels, | Osteotome, |
| | visceral ganglia | myotome, |
| | | dermatome |
| Forced posture | No | Yes |
| Sleeping | Always disturbed | Falling asleep is |
| | | difficult |
| Pain syndrome pattern | Sudden, | Usually is |
| | paroxysmal | permanent |
| Emotional feelings | Fear is common | No fear |
| Analgesics efficiency | Low | Helpful for |
| | | certain period |
| Opiates efficiency | Reduces but does | Eliminates pain |
| | not eliminate the | |
| | pain | |
| Antispasmodics/smooth | Temporarily | Not effective |
| muscle relaxants | effective | |

The difference helps us to recognize if the hernia has occurred and enzyme leakage brought the irritation to an advanced stage. The irritation spreads to the autonomic nervous system, and the pain-pattern usually gets changed.

NES (neurotome electrical stimulation) is a treatment of the involved neurotome, by indirect stimulation via osteotome and dermatome. Indications, that such stimulation is required are when the patient complains of a painful vertebra and distal somatic neuralgic pain, with its original source at the vertebral column. Feelings of pain are often described as burning, pressure, twisting, or even dizziness. Medical investigation, and, perhaps, subsequent disc treatment may be required due to the rupture of the nucleus pulpous.

The main purpose of this treatment is to send electrical impulses through the dorsal root and lower the motor neuron to the distal part of the nerve (skin endings). The pointed entrance and disperse, exit at the determined area to effectively employ the nerve without touching it. The secondary benefit is a contraction of muscles which helps release nerve compressed by strained muscles. As the stimulation is confirmed, we may observe a contraction of muscles innervated by the nerve branch. The treatment is most beneficial for cases like sciatica or carpal tunnel syndrome, as described below.

Osteotome Electrical Stimulation (OES).

OES is designed to stimulate muscles and other soft tissues (as well as the vertebra itself) in close contact with the involved vertebra. The need for such treatment is based on the concept that vertebra-supporting muscles, of any size, play a significant role in the alignment of the spine. Therefore, long-term backache almost always means a displaced and sore vertebra. Finger pressure applied on such a vertebra will produce discomfort and pain. Sets of such treatments will relieve back pain and improve the paravertebral muscle condition. In addition, some patients got their spine alignment back without any vertebra manipulation.

A negative electrode (needle) is placed on the periosteum of the vertebra. The needle does not need to go inside the bone. In fact, as soon as the needle contacts the periosteum, the stimulation may start. A positive electrode (an electro-conductive patch) is placed on a front area within the boundaries of the dorsal root. The treatment looks like a neurotome stimulation but actually serves another purpose. An absence of a major nerve between two electrodes will create a stronger electrical field in the area of the needle tip, and will transmit it to surrounding muscles and soft tissues. Strong pounding of spinal muscles will confirm a well-installed treatment.

Treatment for Low Back Pain

Lower back pain is the most frequent malady, affecting one in three people over the age of 45. Most frequently affected areas are the L4-5 and S1-2 vertebrae.

During an injury, a part of a muscle is excluded from the performance. Usage of the injured muscle is painful, and limits body movement, which creates a specific posture. The posture usually brings unequal biomechanical pressure on the side of the vertebra. The unequal pressure on a disk, over a long period, creates cracks, or a hernia, which can compress a nerve branch and develop into sciatica. The hernia situation may only be solved by surgery, or disk treatment, with an elimination of the cause of irritation.

The Clinical Symptoms Characterized by Somato-sensory Feeling.

The patient with a sharp pain and no somato-sensory feelings does not fit the category, because the nerve branch may be pinched, however at a lower level. It means the nerve is pinched inside, or between muscles. This situation often confuses some practitioners. The Merck Manual, states, “â□the pain most commonly caused by peripheral nerve root compression is from intra-vertebral disk protrusion or intra spinalâ□The nerves can also be compressed outside the vertebral column, in the pelvis or buttocks.”(Section 5) Nobody doubts it, but I repeat, many doctors are convinced that 85-90% of backache originates from soft tissues, and, therefore, may be unnecessarily conservative in their treatment of the patient.

General overlook: such signs as asymmetry in the hips or thighs, hollow back, overweight, and scoliosis, help us recognize what muscles or side of the body are more vulnerable to muscle strain. Therefore, patients who mention the above pre-existing conditions need more prolonged initial sessions, and a clear understanding of their condition, which requires preventative treatment in the future.

The next step is an examination of the troubled area: Palpitate and recognize every sore spot in the troubled area. Watch for the direction of the examined muscle; it will help to recognize the muscle. Memorize or mark them. There are always a few of them. Palpitate and recognize sore vertebrae when finger pressure is applied. Mark them. Relate sore spots and sore vertebrae. Choose one or two of the most sensitive vertebrae for the pressure treatment.

The treatment's goal is usually divided into two parts:

Part 1) Stimulation of the vertebrae

Part 2) Stimulation of sore spots (muscles) of the area

Part 1) Stimulation of the vertebrae, means the stimulation of all deep muscles (semispinalis, multifidus, rotatores) that support the vertebrae. Every strain starts from part of the muscle, and if untreated or aggravated future, will spread over a wider area, ultimately involving other muscles. This condition happens because of the impaired ability of the injured muscle to contract, causing neighboring muscles to take over its function. The more time that patients go without treatment, the more chances of involvement of deep muscles/tendons. The stimulation of palpated sore muscles is usually includes sore spots on the iliocostalis lumborum, longissimus dorsi, spinalis dorsi or quadratus lumborum.

a) To place a needle on a process spinous

b) To find a place for a patch.

The area between the navel and pubic bone will support electrical stimulation with the needle placed on the vertebrae from L4-5 to S1-2. Practice tells us that this area will support electrical stimulation, even wider, using vertebrae L2-3 to S2-3, due to the metamere overlapping.

A patch is more convenient than a needle on the belly for two reasons. It is less painful, and it supports more powerful stimulation of an active needle on the back.

After a stimulation of deep tissues surrounding a vertebra, we have to provide treatment for sore muscles, which the practitioner has to find, localize, recognize and provide the stimulation to these muscles.

An Example of Treatment for an Average Low Back Pain (Osteotome Stimulation).

A needle is placed on the process spinous of L4 and a patch on CV3 (acu-point which is located on 1 inch above edge of pubic bone. The size of the needle for an average patient is 0.35 (30 mm). Electrical frequency is two Hz, with an intensity of 15-25 milliamps. The time for stimulation is 7-15 min. The red (negative) electrode goes on a vertebra and the black (positive) attaches to a patch.

Note: the patient will experience slight feelings of discomfort, and powerful contractions of the stomach, if there are large fat deposits on the stomach, scars from previous surgery, or the needle did not reach the periosteum. Try to avoid this as much as possible, and do not place a patch on a scar—for example, when scars and recommended treatment spots are the same. The session will be more profound if the patch is moved above the scar proximally or laterally.

After the process of locating sore muscles, stimulation may be induced by placing one needle on a vertebra and placing another on the injured muscle. The best scenario is when the same needle is employed for both parts one and two, avoiding unnecessary pain to the patient. The “muscle's needle” has to reach the sore spot. To do this correctly the practitioner must calculate the depth of needle insertion, using his knowledge of anatomy and estimation of the obesity of the patient.

Preferably, the needle is inserted in the middle of the muscle. The size of the needle for an average patient is 0.30×35 mm. The needle for the vertebrae has to be thicker, to avoid misguiding. In this case, the needle size, 0.30×35 mm is optimal. The polarity is always the same—red on a vertebra and black on the patch or muscle.

An Example of the Treatment Session.

Place one needle on L4, with the same precautions, and the other one on the painful area of the longissimus dorsi, iliocostalis lumborum, quadratus lumborum, gluteus medium, or gluteus maximus. The electrical frequency should be two Hz, and the intensity should be 15-25 milliamps. This is considered safe, and the “muscle”needle may be inserted as deep as it is needed. A 0.30×75 mm needle is usually large enough even for obese patients. The time for stimulation is 7-10 minutes.

Sciatica.

Sciatica is usually a complication of untreated low back pain. From my observations, patients seem to get pain in the lower back first. If they continue with the same lifestyle, or type of work, and don't do anything to prevent worsening their condition, they will develop sciatica signs, or sciatica, because the nerve is being pinched between swollen muscles. (A herniated disk is a different problem and we do not discuss it now.) The result depends on choosing the correct vertebra for stimulation. Stimulation provided above, or especially below, the injured area will be less effective.

The Treatment for the Average Sciatica.

Neurotome stimulation. Localize the most sensitive vertebra and place a needle on it. The needle is supposed to be attached tightly to the bone part or even slightly inserted. An active electrode is attached to the vertebra and a patch on acu-point B57.

A patch works better than a needle because the affected area involves several large nerves and more than one metamere. Point B-57 is related to the bladder channel that is responsible for the low back pain condition. Classical sciatica is usually noted as shooting pain in the direction of the heel, by the leg posterior midline. The patch location should be different if the pain goes in another direction.

The intensity of the stimulation depends on the precise needle insertion and obesity level of the patient. The best scenario is when the contraction is very obvious and painless. The needle size, 0.30×30 mm, is good for an average person. Frequency is one-two Hz and intensity is 25-40 milliamps. The time for stimulation is 7-15 minutes.

Myotome stimulation. Sciatica nerve may be compressed by strained hip's muscles like Gluteus medius, piriformis, maximus, obturator internus and gemelli.

The needling is more complicated because for access to priformis obturator internus and gemilli the patient has be on healthy side with affected limb knee bent and brought to a stomach as much as possible.

Shoulder and Neck Pain.

Most shoulder pain can be treated by applying a needle (passive) on the C7th vertebra, and a needle (active) on a sore muscle. The intensity may be less than for back treatment because of the smaller size of the neck/shoulder muscle and the distance between these electrodes. The practitioner must be aware of the depth of the needle insertion into the muscles. The depth depends on obesity level, anatomical location, and size of the muscle. The needle length, 3-3.5 sm, is for an average person and not for a child or an obese client. The practitioner's common sense is the best guide.

A needle must be applied only into the muscle tissue. So palpation of the area is mandatory to avoid puncturing major blood vessels, nerves, or lungs. The finger must be placed on the muscle, and the practitioner must be sure that the needle goes into the desirable target. Three rules must be applied for any sessions: 1) The target muscle must be hit.

2) The session-must be comfortable. There should only be muscle stimulation and contraction, not pain.

3) The practitioner must know how far to insert the needle.

Special considerations should be taken for treating muscles of the lung apex-area such as the levator scapulae, scalenius, and serratus anterior. A thicker “vertebra”needle such as a 0.35-0.40 mm. and shorter like 3.5 sm is preferable for safety reasons. A thin needle is much more flexible; therefore, it will not always go to the desired point.

Evaluation of the ROM of the neck is necessary to locate the proper area of treatment, side of the neck, layer of a neck muscles. Injuries of the above mentioned muscles are mostly related to MVA (whiplash injury) and require the treatment of a more experienced practitioner than, for example, one who would only treat low back pain.

Treatment for Average Neck or Shoulder Pain

A needle on the C5 vertabra, another needle on the levator scapulae, trapezius, or longissimus capiti.

The frequency 2 Hz, the voltage 15-25 milliamps, the time is 5-10 minThe best way is to needle another muscle once you finish stimulating the previous one. In this way, the patient will have only two needles in him at the same time. The muscle on the front of the neck, i.e. levator scapulae, requires less intensive stimulation, often two-five milliamps.

Chest Pain.

The majority of patients seek consultation from a doctor because they are in pain, which is preventing them from working, or otherwise enjoying their life. Complaints of chest pain are special, because it usually makes the patient concerned about their heart.

If the patients are in the 40-60 age group, chest pain almost always makes them think about a possible heart attack.

Not every family physician is able to successfully diagnose chest pain without necessary tools. The usual scenariois for the doctor to order blood tests and an ECG. At this point, the patient is a little less anxious because he/she is under observation. Often, the doctor will reach the conclusion that the case is not medical, “it is just a muscle pain.”In this case, the pain usually originates from an area beside vertebra TH3-6, or most common, TH 4-5, on spinalis or semispinalis. The swollen or inflamed muscle irritates the intercostals nerve, and pain radiates toward the nerve ending—sternum. Precise locating and recognition of the sore spot is an important element of successful treatment.

Treatment: Vertebrae needle is on C7-Th1, while the muscle needle is on the sore spot of the semispinalis or spinalis muscle. Intensity is 15-20 milliamps, frequency is one Hz, duration is 7-10 minutes.

Upper Neck.

The muscle-strain injury in the suboccipital region/triangle plays more significant role in causing headache, insomnia, and dizziness than we think.

Another area requiring a more skilled practitioner is the upper neck. From my experience, I've learned that muscle injury of the suboccipital triangle, made by obliqus capitis inferior, obliquus capitis superior, and rectus capitis posterior major, are often related to headaches, dizziness, insomnia, forgetfulness, and a “not-well-being” feeling. The severity of these complaints relates to the type and age of the injury. The greater occipital nerve and vertebral artery may have influenced the shortened muscles, or put pressure on the swollen musclesThe most frequently damaged muscle is obliquus capitis inferior.

The treatment: a needle is placed on C2. The needle has to be short and thick to insure it goes into the process spinous. Needling of the muscle, obliquus capitis inferior, requires precise skills, knowledge of anatomy, and the insertion must be done under the left thumb (if you right handed) control. The practitioner locates the muscle and needles it at once without moving the thumb. One hand does the locating, and the other inserts the needle. This process is the best way to control the action.

The procedure frequency and intensity are same. A few muscles like are levator scapulae and scalenius medius are required much less electrical intensity than other—3-7 mAmps.

There is a difference in treating MSI, like whiplash, and any RSI of the neck area. RSI is usually easier to locate because the injury has developed over a prolonged period of time, and injured muscles/muscles-threads are grouped together. Whiplash is more difficult to locate on palpation. Range of movement is more informative and helps to focus on more impaired muscles. Due to the excessive but short-time impact, muscle-threads get ruptured and the injury is wwidespread And, of course, it also depends on the power and direction of the impact, as well as the head position at impact time. Guidelines for these particular cases are the same but improvement is slower.

Carpal Tunnel Syndrome.

Carpal Tunnel is deep groove on the front of the carpal bones roofed with Transverse Carpal Ligamentand converted into a tunnel, through which the Flexor tendons of the digits and the median nerve pass. Symptoms of median nerve compression due reduced room at Carpal tunnel is called Carpal tunnel syndrome. The recent researches prove that thickened sheath of tendons is a one of key factors of the impairment. The reasons may be are sclerotic process and inflammation/edema. The sclerotic process of synovial layer is usually irreversible. The treatment is surgical. The treatment offered here is proved effective for Carpal tunnel syndrome related to repetitive strain injuries and on pre-surgical stage only.

Statistics tells that women have the problem more often than men. The common thing among women, when they have carpal tunnel syndrome on one hand, is that over a period of the time it occurs on the other hand. The time may vary from a few months to a few years. Often it correlates with pregnancy and edema, and as soon the pregnancy/edema is over, the symptoms disappear.

Chiropractic doctors believe than carpal tunnel syndrome develops due to a neck condition. Successful neck manipulation improves some patient's condition.

The treatment is based on the idea that carpal tunnel space reduces due inflammation/edema of belonging tissues like muscles and tendons. The carpal tunnel-syndrome may be also a manifestation of a distant injury of neck muscles/vertebra. Therefore the treatment area supposes include neck, arm and wrist. When electrical impulses are sent through the periosteum and lower motor neuron to the wrist the stimulation includes all three levels—osteotome, myotome, and neurotome. The application of a needle on affected vertebra, which is often C 4-5, and a patch on a base of palm, just below carpal tunnel support the requirements of the stimulation. The frequency is 2-3 Hz, intensity is 25-40 mAmps and the stimulation time is 10 min. Amount of required sessions relays on impairment age. The longer the patient delays the treatment the more sclerotic tissues build up, the more reasons to go for surgery than any conservative treatment.

Pain in the Sole.

This is a very common malady among runners and some ladies over 40 years of age. This injury is most often related to the flexor hallucis longus, as is presented as semi-tendinous muscle on the plantar surface. It goes from the big toe toward the calcaneus, by the medial side. Shortening of the tendon causes painful walking, as well as deviation of the big toe over a long period of time.

Treatment: applying a needle spinous process on L5, and another needle on the muscle. The best way for needling is to have foot at the dorsiflexion position. Stretch the muscle, and insert the needle at the most sensitive spot, making sure the needle has reached the spot. Let the patient gently relax the foot, connect the wires to the needle and and stimulate it for 7-10 minutes, at an intensity 20-30 milliamps and, a frequency of two Hz.

Injuries Related to Excessive Jogging.

Usually, athletes suffer calf pain. Palpation of the calf should be done when the foot is at the dorsiflexion position. Common area of injuries are:1) Back lateral/medial side of calf and 2) Lateral/media-lateral side of knee. The injured muscles usually are flexor hallucis longus, which may be combined with the flexor digitorum longus and tibialis posterior.

Treatment: The foot must be at the dorsiflexion position for flexors muscles, before treatment, at needling or palpation time. Position for the “vertebra”needle is S2 spinous process. The frequency is two to three Hz, intensity should be 25-30 milliamps, for seven to ten minutes for every muscle.

Knee Pain.

Muscle-related knee pain is usually found in the distal part of the vastus lateralis and vastus medialis. However, stimulation of the near-to-knee muscle part is complicated. The best way to do this is to insert the needle into the middle part of the muscle without paying attention to the sore-spot location. It is difficult to obtain efficient contraction from any near-to-knee muscle part.

The vertebra needle position is L3 spinous process. The frequency is two to three Hz, and the intensity is 25-30 milliamps. Time required for stimulation is seven to ten minutes.

1. A complex of methods of treating skeletal muscle strain injury and other injury's involved soft tissue comprising:
1.1 Myotome electrical stimulation for a muscle strain injury: A method of employing a whole muscle contraction by electrical intramuscular stimulation with a frequency 1-2 Hz and intensity 5-35 mAmp.
(A) Locating an injured muscle and inserting an acupuncture needle into the muscle.
(B) Recognize lower motor neuron related to the muscle and placing an acupuncture needle on a vertebra's periosteum related to the lower motor neuron.
(C) Providing electrical stimulation for obtaining obvious contraction of a desired muscle by employing lower motor neuron
(D) Withdrawing said needle from a muscle after 5-7 min of stimulation and repeating steps (A) and (D) of another injured muscle if it is within boundaries of same myotome. Repeat step (A), (B), (C) and (D) if the other muscle is out of area of the myotome.
1.2 Neurotome electrical stimulation/treatment for an impaired nerve branch (carpal tunnel syndrome, sciatica etc.).
Frequency is 1-2 Hz and intensity is 5-55 mAmp.
(A) Placing a needle on involved (most sensitive) vertebra and establishing contact between the needle and the periosteum.
(B) Placing an electro-conductive patch on distal area of involved nerve branch
(C) Providing electrical stimulation on involved neurotome for 7-10 min
(D) Removing needle and patch or repeating steps (A), (C) and (D) from 1.1 if any injured muscle at area of the neurotome.
1.3 Osteotome electrical stimulation for an involved vertebra, mostly of lumbar and sacral area (low back pain) and intrinsic muscle.
(A) Placing a needle on involved vertebra (source of pain) and establishing contact between the needle and the periosteum.
B) Placing an electro-conductive patch on middle frontal line (an abdominal wall in a case of low back pain) within osteotome boundaries of the vertebra
(C) Providing an electrical stimulation and obtain obvious low back muscles contraction for 5-7 min. Muscle contraction of an abdominal wall is not desirable but difficult to avoid.
(D) Removing needle and patch or repeating steps (A), (C) and (D) from 1.1 if any injured muscle at area of the neurotome.

2. A method according to claim 1 (steps (A) and (B)), wherein said a needle is held stationary while said stimulation is provided.

3. A method according to claim 1.1, wherein steps (A) through (D) are repeated to elicit stimulation/contraction on any affected muscle upon limit of treatment time.

4. A method according to claim 1.2, wherein steps (A) through (D) are repeated to elicit stimulation on any affected muscle upon limit of treatment time.

5. A method according to claim 1.3, wherein steps (A) through (D) are repeated to elicit stimulation/contraction on any affected soft tissue in close contact with affected vertebra.

6. A method according to claim 1.1, 1.2 and 1.3, wherein said time period of contraction is in the range of 5-10 min.

7. A method according to claim 1.1. 1.2 and 1.3 wherein a stimulation is performed by single impulse (normal mode) with the frequency in the range of 1-3 Hz.

8. A method according to claim 1.1, wherein the muscle is exposed to a current density of approximately 5-35 mAmp.

9. A method according to claim 1.2/1.3, wherein the neurotome/osteotome is exposed to a current density of approximately 20-60 mA., which is highly depends on health condition of the vertebra and distance between the needle and the patch.

11. A method according to claim 10 wherein said electrical current is permanent one.

12. A method according to claim 10 wherein said permanent current is fixed amplitude permanent current.

13. A method according to claim 10, wherein the permanent current is in the form of a short (0.4-200 microsec) monophasic/biphasic square wave.

14. A method according to claim 1.1, 1.2 and 1.3 wherein said a negative electrode (needle) is always placed on a vertebra (spine) and a positive electrode (needle or patch) on a more remote part of body regarding a spine and in the boundaries of the metamer.e

15. A method according to claim 1.1, 1.2 and 1.3 wherein in step (B) the needle is inserted into the patient's vertebra generally in an attachment with the periosteium.

16. A method of treatment is based on conception of muscle injury as a complex process of intramuscular swelling, accumulated metabolic by products, impaired blood circulation and compression of intramuscular nerve contrary to other treatments where conception is based on muscle spasm theory.

17. A method of eliciting muscle contraction by employing a vertebra's periosteum and spinal nerve as a mediator between two electrodes.
